(19) United States
(12) Patent Application Publication
(10) Pub. No.: US 2009/0214648 A1
(43) Pub. Date: Aug. 27, 2009

(54) PHARMACEUTICAL FORMULATIONS COMPRISING IBUPROFEN AND DIPHENHYDRAMINE

(76) Inventors: Malathi Kandakatla, Hyderabad (IN); Dinesh Dayaramji Chakole, Nagpur (IN); Pradeep Kumar Reddy Chellekkagari, Hyderabad (IN); Venkata Sivareddy Pallempalli, Hyderabad (IN); Raviraj Sukumar Pillai, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/370,955

(22) Filed: Feb. 13, 2009

Related U.S. Application Data
(60) Provisional application No. 61/081,231, filed on Jul. 16, 2008.

(30) Foreign Application Priority Data
Feb. 15, 2008 (IN) 389/CHE/2008

(51) Int. Cl. A61K 9/32; A61K 31/192; A61K 9/28

(52) U.S. Cl. 424/482; 514/570; 424/474

(57) ABSTRACT

A pharmaceutical dosage form comprising diphenhydramine, or a pharmaceutically acceptable salt thereof, and ibuprofen, wherein the dosage form provides both diphenhydramine and ibuprofen in a single monolayer tablet, and processes for preparation of dosage forms.

The present invention relates to pharmaceutical formulations containing ibuprofen and diphenhydramine, including its pharmaceutically acceptable derivatives and mixtures thereof. The invention also relates to processes for preparing the pharmaceutical formulations and methods of using such formulations. Further, an aspect of the invention relates to stable pharmaceutical formulations containing ibuprofen and diphenhydramine, including its pharmaceutically acceptable derivatives and mixtures thereof.

Ibuprofen is therapeutically used as an analgesic and non-steroidal anti-inflammatory drug (NSAID). It is indicated for treating musculoskeletal pain, osteoarthritis, rheumatoid arthritis, and juvenile chronic arthritis. Ibuprofen has prominent anti-inflammatory effects in addition to having analgesic and antipyretic actions. The analgesic effects of ibuprofen are due to both a peripheral and a central effect, and are distinct from its property as an anti-inflammatory drug. Ibuprofen is a potent inhibitor of the enzyme cyclooxygenase, which results in a marked reduction in prostaglandin synthesis.

Ibuprofen is a white powder or crystalline solid with slight odor and taste. It is practically insoluble in water and is soluble in alcohol, chloroform, acetone, methanol, ether and dichloromethane. Chemically, it is a propionic acid derivative and has a chemical name 2-[4-(2-methylpropyl)phenyl]propanoic acid. Ibuprofen is represented by structural Formula 1.

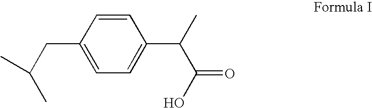

Following oral administration, ibuprofen is rapidly and almost completely absorbed. Peak serum levels are achieved between 1 and 2 hours after dosing. Ibuprofen is rapidly eliminated from the plasma, with half-life of about 2 hours, except at high concentrations; about 99% of ibuprofen is bound to plasma albumin, and this results in a relatively low volume of distribution, about 0.1 L/kg. Ibuprofen is extensively metabolized in the liver, with more than 90% of the dose excreted in the urine and remainder presumably in the faeces. Less than 10% of the dose is excreted unchanged. Excretion is essentially complete within 24 hours.

Diphenhydramine, an ethanolamine derivative, is a sedating antihistamine, H1 receptor antagonist, having antimuscarinic, sedative, anticholinergic, antiemetic, antitussive, anti-motion-sickness, central nervous system (CNS) excitation and CNS depression properties.

Diphenhydramine citrate is white crystalline odourless powder, sparingly soluble in water. It has chemical names ethanamine, 2-(diphenylmethoxy)-N,N-dimethylamine and [2-(diphenylmethoxy)ethyl]dimethylamine. Commonly used salts of diphenhydramine include diphenhydramine hydrochloride and diphenhydramine citrate. Diphenhydramine citrate has chemical names 2-(diphenylmethoxy)-N,N-dimethyl-,2-hydroxy-1,2,3-propane tricarboxylate (1:1), or 2-(Diphenylmethoxy)-N,N-dimethylethylamine citrate (1:1). Diphenhydramine citrate is structurally represented by structural Formula II.

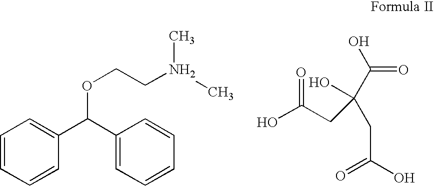

Diphenhydramine is well absorbed following oral administration. Following oral administration of a single dose of a diphenhydramine salt, the drug appears in plasma within 15 minutes, and peak plasma concentrations are attained within 1.5-4 hours. The usual dose of diphenhydramine is 25 mg to 50 mg, three or four times daily.

Combinations of ibuprofen and diphenhydramine are commercially available as soft gelatin capsules and caplets. ADVIL® PM Liqui-Gels® soft gelatin capsules contain 25 mg diphenhydramine hydrochloride and 200 mg ibuprofen, and ADVIL® PM caplets contain diphenhydramine citrate 38 mg and 200 mg ibuprofen. The ADVIL PM product is a film-coated bilayer caplet, where ibuprofen is present in one layer and diphenhydramine citrate is present in the other layer.

U.S. Pat. Nos. 5,431,916, 5,512,300, 6,251,426 and 6,287,600 describe combinations of diphenhydramine with various analgesic, anti-inflammatory drugs and methods of eliciting enhanced analgesic and anti-inflammatory response.

U.S. Pat. No. 4,522,826 describes the effect of ibuprofen and diphenhydramine and methods of eliciting enhanced analgesic and anti-inflammatory response.

U.S. Patent Application Publication Nos. 2002/0177626 and 2007/0003622, and International Application Publication Nos. WO 2005/120465 and WO 2005/079272 teach formulations comprising diphenhydramine and ibuprofen.

Some of these applications have disclosed that ibuprofen and diphenhydramine tend to react with each other when placed together. Therefore there is a need to stabilize formulations containing ibuprofen and diphenhydramine.

### SUMMARY

The present invention relates to pharmaceutical formulations comprising ibuprofen and diphenhydramine, including its pharmaceutically acceptable derivatives and mixtures thereof.

In an embodiment the present invention provides stable pharmaceutical formulations comprising ibuprofen and diphenhydramine, including its pharmaceutically acceptable derivatives and mixtures thereof. In embodiments the invention provides stable pharmaceutical formulations comprising ibuprofen and diphenhydramine or its salts, wherein total drug-related impurities in the formulations are not more than about 2%, or not more than about 1%, by weight of the formulation.

In embodiments, levels of drug-related impurities derived from the two active ingredients is not more than about 0.5%, by weight of the total formulation.

In one of the embodiments, the present invention includes formulations having moisture contents, which are not more than about 5% w/w.

In another embodiment the present invention relates to pharmaceutical formulations containing ibuprofen and diphenhydramine wherein both the active ingredients are compounded together in a solid dosage form.

In an embodiment the present invention relates to pharmaceutical formulations comprising ibuprofen and diphenhydramine citrate.

In another embodiment the present invention relates to stable pharmaceutical formulations comprising ibuprofen and diphenhydramine citrate.

In one embodiment the present invention comprises processes for preparing pharmaceutical formulations comprising ibuprofen and diphenhydramine or its salts.

In another embodiment the present invention comprises processes for preparing pharmaceutical formulations wherein ibuprofen and diphenhydramine or its salts are processed in dry conditions.

In a embodiment the present invention relates to methods of using the pharmaceutical formulations in treating pain associated with sleep disturbances and enhanced analgesic and anti-inflammatory effects, as compared with ibuprofen alone.

### DETAILED DESCRIPTION

The present invention relates to pharmaceutical formulations comprising ibuprofen and diphenhydramine, including its pharmaceutically acceptable derivatives and mixtures thereof.

In an embodiment of the present invention, a unit dose of ibuprofen ranges from about 1 mg to about 400 mg, or about 100 mg to about 300 mg, or about 150 mg to about 200 mg.

In the present invention, pharmaceutically acceptable derivatives of diphenhydramine include but are not limited to salts, polymorphs, solvates, esters, hydrates, enantiomers, racemic compounds, and mixtures thereof.

In one embodiment a salt of diphenhydramine is diphenhydramine citrate. A unit dose of diphenhydramine citrate in the present invention ranges from about 1 mg to about 60 mg, or from about 20 mg to about 40 mg.

Solid ibuprofen and diphenhydramine compositions of the present invention can be filled into capsules or can be compressed into tablets.

In embodiments, pharmaceutical formulations of the present invention are prepared in such a way that ibuprofen and diphenhydramine citrate are compounded together into a solid dosage form, and are not physically separated from each other.

In one aspect of the present invention, the two active ingredients are intimately mixed and are subjected to stability studies at accelerated testing conditions of 40° C. and 75% relative humidity (RH) for a period of three months. The samples are evaluated periodically using X-ray diffraction and impurity analyses. The samples analyzed are found to be stable.

In one aspect of the present invention pharmaceutical formulations have moisture contents not more than about 5% by weight. The presence of water can be responsible for promoting undesired interactions between ibuprofen and diphenhydramine.

In embodiments of the present invention, pharmaceutical formulations containing ibuprofen and diphenhydramine have limited concentrations of the following impurities related to ibuprofen:

2-(4-Formylphenyl) propionic acid (“FPPA”), represented by Formula III.

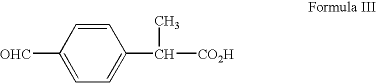

2-(4-Methylphenyl) propionic acid (“MPPA”), represented by Formula IV.

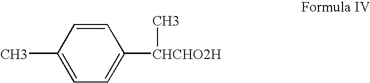

2-(4-Isobutylphenyl) propionic acid (“IBPA”), represented by Formula V.

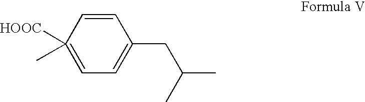

2-(4-Ethylphenyl) propionic acid (“EBPA”), represented by Formula VI.

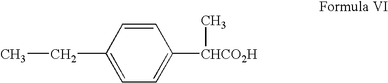

4-Isobutylacetophenone (“4-IBAP”) (Ibuprofen related compound C), represented by Formula VII.

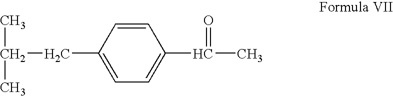

2-(4-n-Propylphenyl) propionic acid (“PPPA”), represented by Formula VIII.

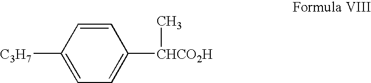

2-(4-n-Butylphenyl) propionic acid (“BPPA”), represented by Formula IX.

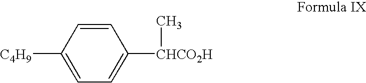

In embodiments of the invention, pharmaceutical formulations of ibuprofen and diphenhydramine, including its pharmaceutically acceptable derivatives, have limited concentrations of the following impurities related to diphenhydramine:

Benzhydrol, represented by Formula X.

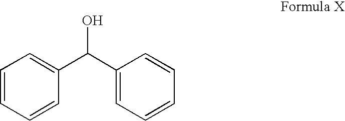

2-(2-(Diphenylmethoxy)-N-methylethylaminethylethylamine (“Impurity A”), represented by Formula XI.

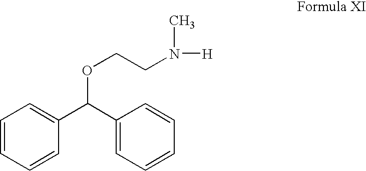

Benzhydryl bromide (“Impurity B”), represented by Formula XII.

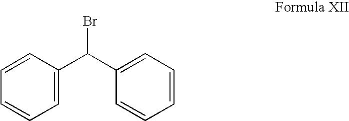

In embodiments, the total concentration of impurities related to ibuprofen and diphenhydramine is less than about 2, or less than about 1, or less than about 0.5, weight percent in a pharmaceutical formulation.

In one of the embodiments of the present invention various parameters impacting compression processes include the physical parameters of the active ingredients as well as that of final component blends, as well as the compactability, flow, and other properties such as moisture content (determined by Karl Fischer (KF) apparatus or infrared moisture balance), particle size (determined by sieve analyzer or a particle size analyzer, such as a laser diffraction instrument sold by Malvern Instruments Ltd., Malvern, Worcestershire, United Kingdom), bulk density and tapped density, compressibility index, Hausner ratio (determined by USP density apparatus), flow properties (determined by Flowdex apparatus), etc.

In an embodiment, the present invention provides stable pharmaceutical formulations comprising ibuprofen and diphenhydramine, including its pharmaceutically acceptable salts, wherein said formulations have moisture contents less than about 8% w/w, or less than about 5% w/w.

In embodiments, bulk densities of final powder blends containing drugs and excipients to prepare pharmaceutical formulations of the present invention range from about 0.2 g/ml to about 0.8 g/ml, or from about 0.4 g/ml to about 0.6 g/ml.

In embodiments, tapped densities of final powder blends containing drugs and excipients to prepare pharmaceutical formulations of the present invention range from about 0.3 g/ml to about 0.9 g/ml, or from about 0.45 g/ml to about 0.7 g/ml.

In embodiments, the invention includes pharmaceutical formulations comprising Ibuprofen with D₉₀ less than about 200 μm, or less than about 125 μm, and diphenhydramine citrate with D₉₀ less than about 150 μm, or less than about 65 μm. D₉₀ is a particle size value, for which 90 percent of the particles in a powder are smaller.

The pharmaceutical formulations of the present invention include dosage forms such as tablets, capsules, pills, granules, pellets, beads and minitablets.

The formulations of the present invention may include any one or more of pharmaceutically acceptable excipients such as diluents, disintegrants, wetting agents, lubricants, and glidants, as appropriate for the intended route of administration. Some suitable excipients are described below.

Various useful diluents include but are not limited to starches, lactose, mannitol, Pearlitol™ SD 200, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as sorbitol and xylitol, calcium carbonate, magnesium carbonate, dibasic calcium phosphate, and tribasic calcium phosphate. In an embodiment the invention includes concentrations of diluents ranging from about 5% w/w to about 85% w/w of the total composition.

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

Useful wetting agents include surfactants like alkyl benzene sulfonate, sodium lauryl sulfate, sodium dilauryl phosphate, lanolin, sodium monoglyceryl lauryl sulfate, sodium methyloleoyl taurate, sodium octoxynol-2-ethanesulfonate, di-β-naphthylmethane disulfate, sulfated castor oil, sodium secondary alcohol sulfate, sodium alkylaryl sulfonate, dialkylsulfosuccinate, dioctyl ester of sodium sulfosuccinate, sodium-7-ethyl-2-methyl-undecyl-4-sulfate, and sodium-3,9-diethyltridecyl-6-sulfate, sorbitan esters, glyceryl monooleate, polyoxyethylene sorbitan fatty acid esters, benzalkonium chloride, benzethonium chloride, cetrimide, polyethylene castor oil derivatives, poloxamers, polyoxyethylene stearates, polyoxyethylene alkylene ethers, stearic acid, lecithin, glyceryl monostearate, cyclodextrins, and benzyl benzoate.

Suitable excipients also may include emulsifying agents such as acacia, anionic emulsifying wax, carbomers, cetosteryl alcohol, cetyl alcohol, cholesterol, diethanolamine, hydrous lanolin, hydroxypropyl cellulose lanolin, lanolin alcohols, methylcelluloses, mineral oil, monobasic sodium phosphate, monoethanolamine, nonionic emulsifying waxes, oleic acid, propylene glycols, propylene glycol alginates and triethanolamine.

Various useful glidants or antisticking agents include but are not limited to talc, silica derivatives, colloidal silicon dioxide, and the like, including mixtures thereof.

Various lubricants that can be used include but are not limited to stearic acid and stearic acid derivatives, such as magnesium stearate, calcium stearate, and zinc stearate, sucrose esters of fatty acids, polyethylene glycols, talc, sodium stearyl fumarate, zinc stearate, castor oils, and waxes.

Various useful film-coating agents include but are not limited to cellulose derivatives such as soluble alkyl- or hydroalkyl-cellulose derivatives such as methylcelluloses, hydroxymethyl celluloses, hydroxyethyl celluloses, hydroxypropyl celluloses, hydroxymethyethyl celluloses, hydroxypropyl methylcelluloses (HPMC, different commercial grades including HPMC 6 cps, HPMC 15 cps, HPMC 50 cps, and others), sodium carboxymethyl celluloses, etc., acidic cellulose derivatives such as cellulose acetate phthalates, cellulose acetate trimellitates and methylhydroxypropylcellulose phthalates, polyvinyl acetate phthalates, etc., insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acid, polyvinylalcohols, polyvinyl acetates, polyvinylpyrrolidones, polymethacrylates and derivatives thereof (Eudragit™), chitosan and derivatives thereof, shellac and derivatives thereof, waxes and fat substances.

If desired, the film coatings may contain additional adjuvants for the coating process such as plasticizers, polishing agents, colorants, pigments, antifoam agents, opacifiers, antisticking agents, and the like.

Various plasticizers include but are not limited to castor oil, diacetylated monoglycerides, dibutyl sebacate, diethyl phthalate, glycerin, polyethylene glycols, propylene glycols, triacetin, and triethyl citrate. Also, mixtures of plasticizers may be utilized. The type of plasticizer depends upon the type of coating agent. A plasticizer is frequently present in an amount ranging from 5% (w/w) to 30% (w/w), based on total weight of the film coating.

An opacifier like titianium dioxide may also be present in an amount ranging from about 10% (w/w) to about 20% (w/w) based on the total weight of the coating. When coloured tablets are desired then the colour is normally applied in the coating. Consequently, colouring agents and pigments may be present in the film coating. Various colouring agents include but not limited to iron oxides, which can be red, yellow, black or blends thereof.

Antiadhesives are frequently used in the film coating process to avoid sticking effects during film formation and drying. An example of an antiadhesive for this purpose is talc. The antiadhesive can be present in the film coating in an amount of about 5% (w/w) to 15% (w/w) based upon the total weight of the coating.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol), and waxes (e.g., carnauba wax, candelilla wax and white wax). In embodiments, polyethylene glycols having molecular weight of 3,000-20,000 are employed.

In addition to the above coating ingredients, sometimes pre-formulated coating material products such as OPADRY™ (supplied by Colorcon), for example Opadry Blue 13B50579, will be used. These products require only mixing with a liquid before use.

Various solvents that can be used in processes of preparing pharmaceutical formulations of the present invention include but are not limited to water, methanol, ethanol, acidified ethanol, acetone, diacetone, polyols, polyethers, oils, esters, alkyl ketones, , methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, dimethylformamide, tetrahydrofuran, and mixtures thereof.

The pharmaceutical formulations may be prepared using any one or more of different formulation techniques such as physical mixing, blending, wet granulation, dry granulation, direct compression, fluid bed granulation, etc. In embodiments, granulation of powders is accomplished using an aqueous solution or dispersion.

An aspect of the present invention is further directed to processes for preparing pharmaceutical formulations comprising ibuprofen and diphenhydramine or salts thereof, wherein an embodiment of a process comprises:

a) Sifting drugs, diluents, disintegrants and other excipients through a sieve.

b) Dry mixing sifted drug, diluents, disintegrants and other excipients.

c) Granulating the dry mix using a binder solution.

d) Drying the granules.

e) Passing the dried granules through a sieve.

f) Mixing the dried granules with sifted extragranular materials and blending.

g) Compressing the blend into tablets.

h) Coating the tablets with a coating dispersion.

In a embodiment the present invention includes either ibuprofen or diphenhydramine or its salt, present in extragranular material.

Pharmaceutical dosage forms can be subjected to in vitro dissolution evaluations according to Test 711 “Dissolution” in United States Pharmacopeia 29, United States Pharmacopeial Convention, Inc, Rockville, Md., 2005 (“USP”) to determine the rate at which the active substance is released from the dosage forms, and the content of active substance can be determined in solutions using techniques such as high performance liquid chromatography.

The pharmaceutical dosage forms of the present invention are intended for oral administration to a patient in need thereof.

In embodiments of the present invention, ibuprofen and diphenhydramine citrate tablets can be packaged in high-density polyethylene (HDPE) bottles and sealed, such as by using a RDE 076 Induction Cap Sealer, from Enercon Industries Corporation, Wisconsin, USA.

The following examples are provided solely to further illustrate certain specific aspects and embodiments of the present invention, and the scope of the invention is not to be limited thereto.

### EXAMPLE 1

### Pharmaceutical Formulation Containing Diphenhydramine 38 mg and Ibuprofen 200 mg

| | |
|---|---|
| Ingredient | Weight Percent |

Granules

| | |
|---|---|
| Ibuprofen | 37.17 |
| Croscarmalose sodium (Ac-di-sol ™) | 1.86 |
| Colloidal silicon dioxide (Aerosil ™ 200) | 0.94 |
| Corn starch | 14.870 |
| Microcrystalline cellulose (Avicel ™ 101) | 13.94 |
| Pre-gelatinized starch (Starch 1500) | 7.44 |
| Sodium lauryl sulphate | 0.46 |
| Water* | q.s. |

Extragranular

| | |
|---|---|
| Diphenhydramine citrate | 7.06 |
| Croscarmalose sodium (Ac-di-sol) | 0.65 |
| Colloidal silicon dioxide (Aerosil 200) | 0.46 |
| Microcrystalline cellulose (Avicel 102) | 13.94 |

Lubrication

| | |
|---|---|
| Stearic acid | 1.21 |
| Total | 100 |

Film Coating

| | |
|---|---|
| Opadry ™ II Blue (49B10882) | 4.83 |
| Water* | q.s. |

*Evaporates during processing.

Manufacturing process:

1. Ibuprofen, croscarmellose sodium, cornstarch, pregelatinized starch, colloidal silicon dioxide and microcrystalline cellulose were sifted through an ASTM #30 mesh sieve.

2. The sifted ingredients of step 1 were blended together in a rapid mixer granulator for about 10 minutes.

3. Sodium lauryl sulphate was dissolved in water with constant stirring to form a solution.

4. Solution of step 3 was slowly added to the dry blend of step 2 over about 2 minutes and mixed together to form a wet mass.

5. The wet mass was dried at 60° C.±5° C. for about 30 minutes in a fluid bed drier until loss on drying was less than 2% w/w.

6. The dried granules were sifted through an ASTM #20 mesh sieve. The retained particles were milled through a 2 mm screen in a multimill and then passed through the #20 mesh sieve and combined with the sifted particles.

7. Diphenhydramine was sifted through an ASTM #24 mesh sieve.

8. Croscarmalose sodium, colloidal silicon dioxide and microcrystalline cellulose were sifted through an ASTM #40 mesh sieve.

9. Ingredients of steps 7 and 8 were blended with the dried granules in a double cone blender for about 15 minutes.

10. Stearic acid was sifted through an ASTM #40 mesh sieve and was blended with the blend formed in step 9 in a double cone blender for 5 minutes.

11. The lubricated blend of step 10 was compressed to form tablets having an average weight of 538 mg.

12. Opadry II Blue 49B10882 was dispersed in water using a mechanical stirrer and the stirring was continued for 45 minutes.

13. The coating suspension formed in step 12 was then coated onto the tablets in a Wurster coater to produce a 4.83% w/w weight build-up.

14. Tablets were dried at 50° C.±5° C. for about 30 minutes.

15. The tablets were packaged in closed HDPE containers.

The packaged tablets prepared above and commercial ADVIL PM caplets were subjected to accelerated stability testing conditions at 40° C. and 75% RH for three months, and samples were analyzed periodically for drug content (expressed as percent of label drug content), drug-related impurity substances and water content (both expressed as percent by weight of the formulation), and drug dissolution characteristics. The results are given below.

| | Example 1 | | | | ADVIL PM | | | |
|---|---|---|---|---|---|---|---|---|
| | | 1 | 2 | 3 | | 1 | 2 | 3 |
| Parameter | Initial | Month | Months | Months | Initial | Month | Months | Months |
| Ibuprofen | 102 | 103 | 104 | 102 | 100 | 101 | 100 | 100 |
| Diphenhydramine | 101 | 101 | 100 | 101 | 98 | 103 | 101 | 98 |
| Highest unidentified | 0.005 | 0.01 | 0.01 | ND | ND | 0.01 | 0.04 | ND |
| ibuprofen-related | | | | | | | | |
| impurity | | | | | | | | |
| Total ibuprofen- | — | — | — | — | 0.01 | 0.01 | 0.06 | 0.01 |
| related impurities | | | | | | | | |
| Diphenhydramine | ND | ND | ND | ND | ND | ND | ND | ND |
| Impurity A | | | | | | | | |
| Diphenhydramine | 0.036 | 0.038 | 0.042 | 0.036 | ND | 0.02 | ND | 0.02 |
| Impurity B | | | | | | | | |
| Highest unidentified | 0.05 | ND | ND | ND | ND | ND | ND | ND |
| diphenhydramine- | | | | | | | | |
| related impurity | | | | | | | | |
| Total impurities | 0.09 | 0.05 | 0.06 | 0.04 | 0.04 | 0.09 | 0.1 | 0.19 |
| Water by Karl Fischer | 3.6 | 3.4 | 3.4 | 4.4 | 4.38 | 4.67 | 6.0 | 4.41 |

Percent of drug dissolved within 45 minutes in pH 7.2
phosphate buffer, 50 rpm stirring, USP type II apparatus

| | | | | | | | | |
|---|---|---|---|---|---|---|---|---|
| Ibuprofen | 99 | 99 | 97 | 99 | 102 | — | 103 | 103 |
| Diphenhydramine | 97 | 99 | 96 | 97 | 100 | — | 98 | 103 |
| citrate | | | | | | | | |

ND = Not detected.

### EXAMPLE 2

### Pharmaceutical Formulation Containing Diphenhydramine 38 mg and Ibuprofen 200 mg

| | |
|---|---|
| Ingredient | Weight Percent |

Granules

| | |
|---|---|
| Ibuprofen | 36.36 |
| Diphenhydramine citrate | 6.91 |
| Croscarmalose sodium (Ac-di-sol) | 1.82 |
| Colloidal silicon dioxide (Aerosil 200) | 0.91 |
| Corn starch | 14.55 |
| Microcrystalline cellulose (Avicel 101) | 27.64 |
| Pre-gelatinized starch (Starch 1500) | 7.28 |
| Sodium lauryl sulphate | 0.46 |
| Water* | q.s. |

Extragranular

| | |
|---|---|
| Colloidal silicon dioxide (Aerosil 200) | 1.82 |
| Croscarmellose sodium (Ac-di-sol) | 0.46 |

Lubrication

| | |
|---|---|
| Stearic acid | 1.82 |
| Total | 100 |

Film coating

| | |
|---|---|
| Opadry ™ Blue (49B10882) | 4.83 |
| Water* | q.s. |

*Evaporates during processing.

Manufacturing process:

1. Ibuprofen, diphenhydramine citrate, croscarmellose sodium, cornstarch, pregelatinized starch, colloidal silicon dioxide and microcrystalline cellulose were sifted through an ASTM #30 mesh sieve.

2. The sifted ingredients of step 1 were blended together in a rapid mixer granulator for about 10 minutes.

3. Sodium lauryl sulphate was dissolved in water with constant stirring to form a solution.

4. Solution of step 3 was slowly added to the dry blend of step 2 over about 2 minutes and mixed together to form a wet mass.

5. The wet mass was dried at 60° C.±5° C. for about 30 minutes in a fluid bed dryer until loss on drying was less than 2% w/w.

6. The dried granules were sifted through an ASTM #20 mesh sieve. The retained particles were milled through a 2 mm screen in a multimill and then passed through the #20 mesh sieve and combined with the sifted particles.

7. Extragranular croscarmellose sodium and colloidal silicon dioxide were sifted through an ASTM #40 mesh sieve.

8. The granules of step 6 and the sifted materials of step 7 were blended together in a double cone blender for about 15 minutes.

9. Stearic acid was sifted through an ASTM #40 mesh sieve and was blended with the blend formed in step 8 in a double cone blender for 5 minutes.

10. The lubricated blend of step 9 was compressed to form tablets having an average weight of 550 mg.

11. Opadry II Blue 49B1 0882 was dispersed in water using a mechanical stirrer and the stirring was continued for 45 minutes.

12. The coating suspension formed in step 11 was then coated onto the tablets in a Wurster coater to produce a 4.83% w/w weight build-up.

13. Tablets were dried at 50° C.±5° C. for about 30 minutes.

The tablets of Example 2 were packaged in HDPE containers and subjected to accelerated stability testing conditions of 40° C. and 75% RH and analyzed for total impurity content initially and at storage intervals. Commercial ADVIL® PM caplets were tested as a reference product. The results are tabulated below, where values are weight percentages of the total formulation.

| Sample | Parameter | Initial | 1 Month | 3 Months |
|---|---|---|---|---|
| ADVIL PM | Total diphenhydramine- | 0.02 | 0.09 | 0.19 |
| | related impurities | | | |
| | Total ibuprofen-related | 0.01 | 0.01 | 0.01 |
| | impurities | | | |
| Example 2 | Total diphenhydramine- | 0.50 | 0.51 | — |
| | related impurities | | | |
| | Total ibuprofen-related | 0.01 | 0.02 | — |
| | impurities | | | |

### EXAMPLE 3

### Pharmaceutical Formulation Containing Diphenhydramine 38 mg and Ibuprofen 200 mg

| | |
|---|---|
| Ingredient | Weight Percent |

Granules

| | |
|---|---|
| Ibuprofen | 37.17 |
| Croscarmalose sodium (Ac-di-sol) | 0.93 |
| Colloidal silicon dioxide (Aerosil 200) | 0.19 |
| Starch | 14.59 |
| Pre-gelatinized starch (Starch 1500) | 3.49 |
| Water* | q.s. |

Extragranular

| | |
|---|---|
| Diphenhydramine citrate | 7.06 |
| Starch | 1.3 |
| Croscarmalose sodium (Ac-di-sol) | 0.93 |
| Microcrystalline cellulose (Avicel 102) | 11.26 |
| Colloidal silicon dioxide (Aerosil 200) | 0.56 |
| Sodium lauryl sulphate | 0.09 |
| Lactose monohydrate (Flowlac ™ 100) | 16.73 |
| Sodium starch glycolate | 0.56 |
| Pre-gelatinized starch (Starch 1500) | 4.09 |

Lubrication

| | |
|---|---|
| Stearic acid | 1.04 |
| Total | 100 |

*Evaporates during processing.

Manufacturing process: similar to that of Example 1.

### EXAMPLE 4

### Pharmaceutical Formulation Containing diphenhydramine 38 mg and Ibuprofen 200 mg

| | |
|---|---|
| Ingredient | Weight Percent |

Granules

| | |
|---|---|
| Ibuprofen | 37.17 |
| Croscarmalose sodium (Ac-di-sol) | 1.86 |
| Colloidal silicon dioxide (Aerosil 200) | 0.93 |
| Starch | 14.87 |
| Microcrystalline cellulose (Avicel 101) | 28.25 |
| Pre-gelatinized starch (Starch 1500) | 7.44 |
| Water* | q.s. |

Extragranular

| | |
|---|---|
| Diphenhydramine citrate | 7.06 |
| Croscarmalose sodium (Ac-di-sol) | 0.65 |
| Colloidal silicon dioxide (Aerosil 200) | 0.46 |
| Sodium lauryl sulphate | 0.09 |

Lubrication

| | |
|---|---|
| Stearic acid | 1.21 |
| Total | 100 |

*Evaporates during processing.

Manufacturing process: similar to that of Example 1.

### EXAMPLE 5

### Pharmaceutical Formulation Containing Diphenhydramine 38 mg and Ibuprofen 200 mg

| | |
|---|---|
| Ingredient | Weight Percent |

Granules

| | |
|---|---|
| Ibuprofen | 37.17 |
| Croscarmalose sodium (Ac-di-sol) | 1.86 |
| Colloidal silicon dioxide (Aerosil 200) | 0.93 |
| Corn starch | 14.87 |
| Microcrystalline cellulose (Avicel 101) | 28.25 |
| Pre-gelatinized starch (Starch 1500) | 7.44 |
| Water* | q.s. |

Extragranular

| | |
|---|---|
| Diphenhydramine citrate | 7.06 |
| Croscarmalose sodium (Ac-di-sol) | 0.65 |
| Colloidal silicon dioxide (Aerosil 200) | 0.46 |
| Sodium lauryl sulphate | 0.09 |

Lubrication

| | |
|---|---|
| Stearic acid | 1.21 |
| Total | 100 |

Film Coating

| | |
|---|---|
| Opadry II Blue (49B10882) | 4.09 |
| Water* | q.s. |

*Evaporates during processing.

Manufacturing process: similar to that of Example 1.

We claim:

1. A pharmaceutical dosage form comprising diphenhydramine, or a pharmaceutically acceptable salt thereof, and ibuprofen, wherein the dosage form provides both diphenhydramine and ibuprofen in a single monolayer tablet.

2. The pharmaceutical dosage form of claim 1, further comprising one or more of a pharmaceutically acceptable diluent, disintegrant, lubricant, binder, glidant, wetting agent, coating agent, or anti-static agent.

3. The pharmaceutical dosage form of claim 2, wherein a diluent comprises microcrystalline cellulose, lactose, mannitol, sorbitol, starch, calcium carbonate, magnesium carbonate, dibasic calcium phosphate, or tribasic calcium phosphate.

4. The pharmaceutical dosage form of claim 2, wherein a disintegrant comprises carmellose calcium, carboxymethylstarch sodium, croscarmellose sodium, a crospovidone, a low-substituted hydroxypropylcellulose, sodium starch glycolate, colloidal silicon dioxide, or starch.

5. The pharmaceutical dosage form of claim 2, wherein a binder comprises a hypromellose, a hyprollose, starch, gelatin, a povidone, or hydroxyethyl cellulose.

6. The pharmaceutical dosage form of claim 2, wherein a wetting agents comprises an alkyl benzene sulfonate, sodium lauryl sulfate, sodium dilauryl phosphate, lanolin, sodium monoglyceryl lauryl sulfate, sodium methyl oleoyl taurate, sodium octoxynol-2-ethanesulfonate, di-β-naphthylmethane disulfate, sulfated castor oil, a sodium secondary alcohol sulfate, a sodium alkylaryl sulfonate, a dialkylsulfosuccinate, dioctyl ester of sodium sulfosuccinate, sodium-7-ethyl-2-methyl-undecyl-4-sulfate, sodium-3,9-diethyltridecyl-6-sulfate, a sorbitan ester, glyceryl monooleate, a polyoxyethylene sorbitan fatty acid ester, benzalkonium chloride, benzethonium chloride, cetrimide, a polyethylene castor oil derivative, a poloxamer, a polyoxyethylene stearate, a polyoxyethylene alkylene ether, stearic acid, lecithin, glyceryl monostearate, a cyclodextrin, or benzyl benzoate.

7. The pharmaceutical dosage form of claim 1, comprising diphenhydramine, or a pharmaceutically acceptable salt thereof, and ibuprofen, combined with at least one excipient and compressed into a tablet.

8. A process for preparation of a tablet, comprising combining: (i) ibuprofen; (ii) diphenhydramine, or a pharmaceutically acceptable salt thereof; and (iii) at least one pharmaceutical excipient; and compressing into a tablet.

9. The process of claim 8, wherein ibuprofen and diphenhydramine, or a pharmaceutically acceptable salt thereof, are present together in granules.

10. The process of claim 9, wherein granules are formed by granulating a powder mixture with an aqueous solution or dispersion comprising a surfactant.

11. The process of claim 8, wherein one of ibuprofen and diphenhydramine or a pharmaceutically acceptable salt thereof is present in granules, and the other is present in an extragranular mixture.

12. The process of claim 11, wherein granules are formed by granulating a powder mixture with an aqueous material.

13. The process of claim 11, wherein granules are formed by granulating a powder mixture with an aqueous solution or dispersion comprising a surfactant.

14. The process of claim 11, wherein ibuprofen is present in granules and a salt of diphenhydramine is present in an extragranular mixture.

15. The process of claim 11, wherein ibuprofen is present in granules and diphenhydramine citrate is present in an extragranular mixture.

16. A pharmaceutical dosage form comprising ibuprofen and a salt of diphenhydramine, wherein ibuprofen is a component of a powder mixture that has been granulated, a salt of diphenhydramine is a component of a powder mixture that is in combination with ibuprofen-containing granules, and the combination is compressed to form tablets.

17. The pharmaceutical dosage form of claim 16, wherein a powder mixture has been granulated with an aqueous material.

18. The pharmaceutical dosage form of claim 16, wherein a powder mixture has been granulated with an aqueous solution or dispersion comprising a surfactant.

19. The pharmaceutical dosage form of claim 16, wherein a salt of diphenhydramine comprises diphenhydramine citrate.

20. The pharmaceutical dosage form of claim 16, wherein a tablet is coated with a polymer film.
